(19) United States
(12) Patent Application Publication
(10) Pub. No.: US 2008/0235853 A1
(43) Pub. Date: Oct. 2, 2008

(54) Heated Earmuff With Improved Frame and Heating Element

(76) Inventor: Kevin James Sousa, Boston, MA (US)

Correspondence Address: WHITE-WELKER & WELKER, LLC, P.O. BOX 199, CLEAR SPRING, MD, 21722-0199, US

(21) Appl. No.: 12/040,947

(22) Filed: Mar. 3, 2008

Related U.S. Application Data
(60) Provisional application No. 60/909,411, filed on Mar. 30, 2007.

(51) Int. Cl. A61F 11/14

(52) U.S. Cl. 2/209

(57) ABSTRACT

The earmuff of the present invention comprises in combination: an adjustable temperature switch, location of a power supply such as a battery inside the earmuff housing, a top frame enhanced to provide a “head gripping” feature, earmuff pads constructed to conform to the ears that are not flat but have some concavity to assist in wearer's comfort and use when moving around. The inner muff is constructed of a soft-foam and further includes a slot that enables the concurrent use of ear phones. The heating element is a plate designed to conserve battery current with adjustable temperature and a circuit design focused on conserving battery current. The plastic frame provides a head gripping feature designed to eliminate unnecessary weight and to maximize air flow that will prevent sweat and moisture buildup around the ears.

### CROSS REFERENCE TO RELATED APPLICATIONS

This application claims priority from U.S. Provisional Patent Application Ser. No. 60/909,411, entitled “Heated Earmuff With Improved Frame and Heating Element”, filed on 30 Mar., 2007.

### FEDERALLY SPONSORED RESEARCH

Not Applicable

### SEQUENCE LISTING OR PROGRAM

Not Applicable

### TECHNICAL FIELD OF THE INVENTION

The present invention relates generally to heated earmuffs. More specifically, the present invention relates to a heated earmuff with an improved frame, efficient heating element and ear pad configuration.

### BACKGROUND OF THE INVENTION

There are many devices taught in the prior art for covering portions of an individual's head for warmth and/or protection from the elements that cover primarily the ears alone. The most common device of ear warming, frequently referred to as an “earmuff,” is comprised of a resilient, C-shaped band or frame that is worn around the top of an individual's head and ear-receiving portions or pockets located at the ends of the C-shaped band.

Typically, an ear warmer comprises a frame and a fabric or foam member. The frame has an ear portion and a band portion. The ear portion of the frame includes a first side and a second side opposite the first side. The first side of the ear portion defines an interior portion of an opening. The second side of the ear portion defines an exterior portion of the opening. The fabric or foam member includes at least its own ear portion coupled to the ear portion of the frame. The ear portion of the fabric or foam member covers the interior portion of the opening in substantially its entirety and covers less than an entirety of the exterior portion of the opening.

Other ear warmers consist of heating devices that may be battery or chemically operated. For example, an exothermic chemically heated ear warmer would include a headband portion and ear covering portions which cover the ears of the wearer and pair of open ended pockets in the ear covering portions which removably receive and carry a packet of exothermic heat dispensing material to warm the ears of the wearer. Other heated ear warmers might include a heating pad placable against the ear and being connected by wiring to either to a household electric outlet or else to a dry cell battery and switch than can be carried in a pocket so the person is thus free to move about either in the home or out-of-doors. These alternative devices are inferior to the present invention as they are cumbersome and restrict a wearer's movement when during any type of activity or motion.

Therefore, it is an objective of the present invention to provide an efficient battery operated heated earmuff with adjustable temperature and means for conserving battery current.

It is another objective of the present invention to teach a frame that provides a head gripping feature designed to eliminate unnecessary weight and to maximize air flow to prevent sweat and moisture build up around the ears.

Still another objective of the present invention is to teach an earmuff that is constructed to conform to the ears and is not flat but has a concavity that assists a wearer in comfort when moving and includes a slot that enables the concurrent use of ear phones under the earmuff.

Another objective of the present invention is to teach an earmuff that is comprised of a heating means that is not cumbersome or cause restricted motion.

Yet another objective of the present invention is to teach an earmuff to efficiently conserve and use battery power to prolong use.

Therefore, current earmuffs do not offer similar construction, comfort, functionality, appearance and flexibility required to adequately provide the features desired by individuals.

### SUMMARY OF THE INVENTION

The present invention is a heated earmuff to help those suffering from chronically cold ears every winter. In a basic embodiment, the present invention is a set of earmuffs which may have any number of coverings from fluffy rabbit fur to a water and wind proof or resistant fabric or foam in combination with a battery operated heating element inside each respective earmuff. In this basic embodiment the present invention uses a wire-based heating element that is similar to those used in a heated blanket or electric socks.

In an improved embodiment, the device comprises in combination an adjustable temperature switch, location of a power supply such as a battery inside the earmuff housing, a top frame enhanced to provide a “head gripping” feature, earmuffs pads constructed to conform to the ears that are not flat but have some concavity to assist in wearers comfort and use when moving around. The inner muff is constructed of a soft-foam or fabric or foam and further includes a slot that enables the concurrent use of ear phones. The heating element is a plate designed to conserve battery current. The nature of the plastic frame will be designed to maximize air flow, which will prevent sweat and moisture build up around the ears.

The primary advantage of the present invention is that it provides an efficient battery operated heated earmuff with adjustable temperature and means for conserving battery current thereby resulting in an extended or longer operating time.

Another advantage of the present invention is an earmuff with a frame that provides a head gripping feature designed to eliminate unnecessary weight and to maximize air flow to prevent sweat and moisture build up around the ears.

Yet another advantage of the present invention is an earmuff constructed to conform to the ears that is not flat but has a concavity that assist a wearer in comfort when moving and includes a slot that enables the concurrent use of ear phones under the earmuff and extended or prolonged use.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is an line drawing illustrating a simple ear warmer device that is well known in the prior art;

FIG. 2 illustrates the contents of an ear pad as taught by the present invention;

FIG. 3 illustrates the improved ear warmer and heating plate of the present invention;

FIG. 4 illustrates a perspective views of the ear warming device from multiple perspectives with respect to the c-shaped frame and ear pads.

### DETAILED DESCRIPTION OF THE INVENTION

In the following detailed description of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention.

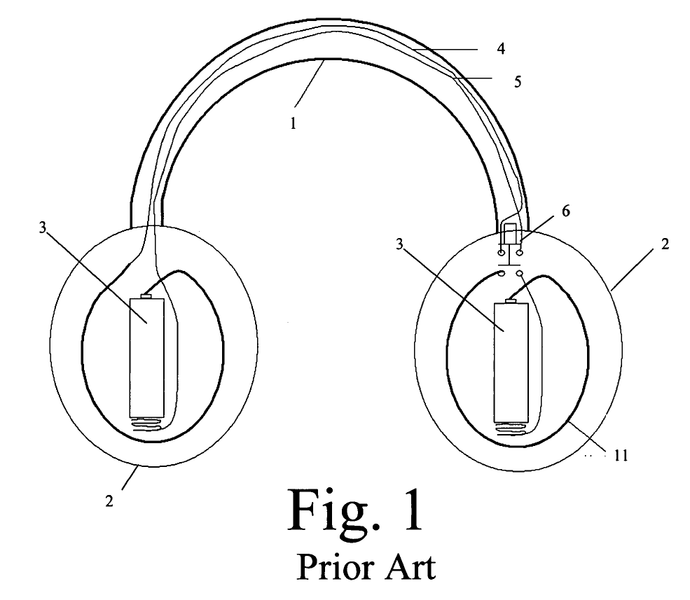

Referring to the figures, it is possible to see the various major elements constituting the apparatus of the present invention. The present invention is an ear warmer comprised of electric heating means and an improved frame. Now referring to FIG. 1, a basic ear warmer known in the prior art is illustrated. Typically, an ear warmer consists of a c-shaped frame 1 with two circular members located at two opposite circumferential edges. Within each circular member 2 is a battery 3 connected by a positive wire 4 and negative wire 5 connected to a simple on/off switch 6 and heating element 11.

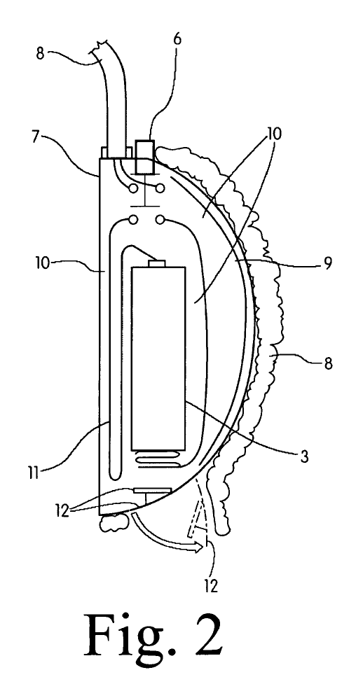

Now referring to FIG. 2, the components of one of two ear pads 7 taught by the present invention is illustrated. In this embodiment the ear pad 7 consists of an external fabric or foam covering 8 that may be of any material readily available in the prior art. Typical fabrics would include fuzzy cotton, or water and wind resistant fabrics. The internal cavity contains a battery 3 as taught by the prior art, but in the present invention, the battery is covered by insulating material 10 which also acts as a safety housing. The insulating material 10 is preferably made from a synthetic or natural fibers. Between the insulating material 10 and the external fabric or foam covering 8 a space blanket 9 layer provides further insulation and protection from the environment. The space blanket 9 is preferably made from a metallized film that reflects heat back into the ear warmer and prevents heat from escaping. One possible metallized film would be biaxially-oriented polyethylene terephthalate (boBET) polyester film, which has shown through testing to be the preferred material for this application. When used in combination the above insulation material and space blanket create a tangled mat that reflects radiant heat and traps heated air while allowing moisture to escape.

The battery 3 is connected by a positive wire 4 and negative wire 5 connected to a rotating variable switch 6 which controls the current distributed to the heating wires 11. Battery 3 access is provided by a removable flap 12 located on ear pad 7.

The actual battery 3 required by the present invention would be that of a standard off the shelf variety common purchased in retail stores. The battery 3 is accessible by the removable flap 12, which may be accessed by a zipper or hook and loop fastener located on the ear pad 7 when the battery 3 requires replacement. The battery 3 will be placed inside a pouch or plastic container and rest inside a pocket located on the ear pad 7 within the ear warmer.

In an alternative embodiment, the battery 3 will be rechargeable with a charging port similar to a cell phone or other portable electronic devices similarly equipped with rechargeable batteries. This will provide a wearer with a simple recharging option and plug as an alternative to continued replacement of the batteries.

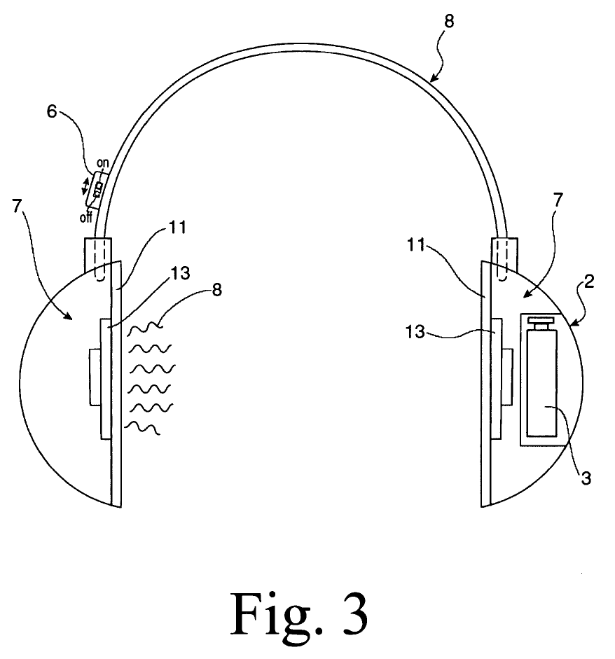

FIG. 3 illustrates an alternative embodiment of the ear warmer of the present invention showing the improved heating element design 13 and position within the ear pad 7. The inner ear warmer surface 11 is constructed of a soft foam or fabric and may be molded with a slot, which will enable the concurrent use of ear phones. The heating plate 13 is designed to conserve battery 3 current. The wires 4 and 5 and materials selected are highly conductive to promote energy conservation. The non-heating wires 4 and 5 are made from small gauge. The heating wires 11 or plate 13 is minimized to provide only an optimal amount of heat.

In one embodiment, the heating plate 13 is in an oval or circular shape to provide a larger heating surface. This larger heating surface uses less heat because it is more efficient at increasing the temperature of an individual's ear by providing a larger area of lower intensity heat in comparison to a heating wire 11 that provides a small area of intense heat, requiring more battery current, leading to decreased battery life and an uncomfortable warming of the ear. The present invention also provides electronic sensors, circuitry, and a microprocessor as means to control current flow in order to prolong battery life and promote comfort to the wearer. The microprocessor provides means for a pulsing current. An HF step-up transformer is used to boost the battery voltage to the heating elements in addition to an encoder for increasing the temperature and an SSR to drive the voltage from the transformer going to the heating element.

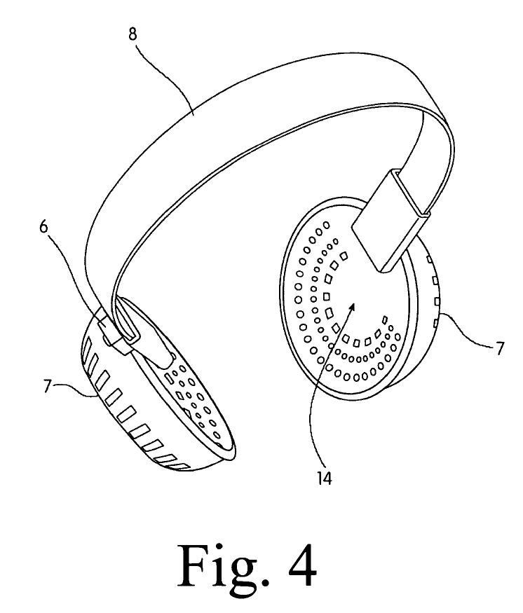

Now referring to FIG. 4, a perspective view of the ear warming device is illustrated. The ear pads 7 are constructed to conform to the ears and are not flat but have some concavity 14 on the inner surface to assist in a wearer's comfort and use when moving around. The top frame 8 is enhanced to provide a “head gripping” feature. The “head gripping” feature is enabled through two different means. The first is the use of materials that have gripping characteristics and the second is the use of a molding that includes small teeth to grip a wearer's head. The nature of the plastic frame 8 is designed with a minimal use of plastic band width and vertical holes in the band to maximize air flow, which will prevent sweat and moisture buildup around the ears. The holes used in the plastic shell, band, and earmuff frame are designed to eliminate unnecessary weight and provide a lightweight design.

Other embodiments include the replacement of the on/off switch 6 with an adjustable temperature switch so a user can adjust the amount of heat being generated by the heating plate 13 to conserve battery power or as desired or required by the temperature of the environment. The battery 3 connected to a rotating switch provides means for regulating the amount of current distributed to a heating element from the battery in order to control heat emission.

Additional electronic switching means may also be used to reduce current loss. The battery may also be rechargeable and incorporate similar technology already known in the prior art for charging cell phone and PDAs via a car or wall plug in charger.

Thus, it is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

The embodiments of the invention in which an exclusive property or privilege is claimed are defined as follows:

1. A warming device for the ears of an individual comprising:
a c-shaped frame;
two circular shell members located at two opposite circumferential edges of the c-shaped frame;
within each circular member there is a battery and heating element; and
said batteries and heating elements are connected to a rotating variable switch which provides means for regulating the amount of current distributed to the heating element from the battery in each respective circular shell member in order to control heat emission.

2. The warming device for the ears of an individual of claim 1 wherein:
the ear pad consists of an external fabric or foam covering;
the ear pad is defined by an interior cavity containing a battery and a heating element;
the battery is covered by insulating material;
a space blanket layer provides further insulation and protection from the environment between the insulating material and the external fabric or foam covering of the ear pad by providing means for reflecting heat back into the earmuff and preventing heat from escaping;
the battery connected to a rotating switch with provides means for regulating the amount of current distributed to a heating element from the battery in order to control heat emission; and
battery access is provided by an removable flap located on the ear pad.

3. The warming device for the ears of an individual of claim 2 wherein the external fabric or foam covering is either cotton or a water and wind resistant fabric or foam.

4. The warming device for the ears of an individual of claim 3 wherein
the inner ear warmer surface is constructed of a soft foam and may be molded with a slot providing means for the concurrent use of ear phones.

5. The warming device for the ears of an individual of claim 3 wherein
the heating element is designed to conserve battery current;
the wires and materials selected are highly conductive to promote energy conservation;
the non-heating wires are made from small gauge;
the heating element is minimized to provide only an optimal amount of heat; and
further comprising electronic sensors, circuitry, and a microprocessor providing means to control the current flow form the battery to the heating element in order to prolong battery life and ensure consistent heating.

6. The warming device for the ears of an individual of claim 3 wherein the heating element is in an oval or circular shape to provide a larger optimal heating surface.

7. The warming device for the ears of an individual of claim 3 wherein the ear pads are constructed to conform to the ears and have some concavity on the inner surface.

8. The warming device for the ears of an individual of claim 3 wherein the top frame 8 is molded to include a plurality of teeth to provide means for gripping a wearer's head.

9. The warming device for the ears of an individual of claim 3 wherein the frame is comprised of a plurality of vent holes in the shell and frame providing means to maximize air flow and prevent sweat and moisture buildup.

10. The warming device for the ears of an individual of claim 3 wherein the device's frame is comprised of a plurality of holes providing means to eliminate unnecessary weight.

11. The warming device for the ears of an individual of claim 3 wherein the rotating switch is adjustable.

12. 11. The warming device for the ears of an individual of claim 3 wherein the rotating switch is non-adjustable.

13. The warming device for the ears of an individual of claim 3 wherein additional electronic switching means may also be used to reduce current loss.

14. The warming device for the ears of an individual of claim 3 wherein the battery is rechargeable via a car or wall plug-in charger.

15. The warming device for the ears of an individual of claim 2 wherein the space blanket is constructed from a metallized film.

16. The warming device for the ears of an individual of claim 15 wherein the space blanket is constructed from biaxially-oriented polyetherylene terephthalate polyester film.

17. The warming device for the ears of an individual of claim 16 wherein the insulating material is either synthetic or natural fiber.

18. The warming device for the ears of an individual of claim 16 wherein the space blanket and insulating material create a tangled mat that reflects radiant heat and traps heated air while allowing moisture to escape.

19. The warming device for the ears of an individual of claim 5 wherein the microprocessor provides means for a pulsing current from the battery to the heating element and electronic circuitry provides means to regulate heat from the heating elements and current loss from the battery.

20. The warming device for the ears of an individual of claim 19 further comprising:
an HF step-up transformer to boost the battery voltage to the heating elements;
an encoder for increasing the temperature; and
an SSR to drive the voltage from the transformer going to the heating element.
